(19) United States
(12) Patent Application Publication
(10) Pub. No.: US 2005/0239744 A1
(43) Pub. Date: Oct. 27, 2005

(54) Method of producing microcrystalline cellulose-containing compositions

(76) Inventors: Michael Ioelovich, Bat-Yam (IL); Alexander Leykin, Haifa (IL)

Correspondence Address: EITAN, PEARL, LATZER & COHEN ZEDEK LLP, 10 ROCKEFELLER PLAZA, SUITE 1001, NEW YORK, NY, 10020, US

(21) Appl. No.: 10/829,460

(22) Filed: Apr. 22, 2004

(51) Int. Cl. A61K 31/716; C08B 1/00

(52) U.S. Cl. 514/57; 536/56

(57) ABSTRACT

A novel, environmentally friendly and resource-saving method for the production of fractionally homogeneous compositions of microcrystalline cellulose (MCC) is provided. The method comprises of the following steps: (a) hydrolyzing cellulose-containing raw material with at least one acidic catalyst in the presence of at least one process additive at a low catalytic system/cellulose ratio; (b) neutralizing said acid with one or more precipitator in a manner that fine particles of insoluble ingredients precipitates into a slurry containing MCC; (c) admixing at least one modifier; following by (d), homogenizing of the composition so that a MCC product characterized by a uniformly dispersed micro-particle cellulose material and various functional ingredients is obtained.

### FIELD OF THE INVENTION

The present invention generally relates to a method of producing microcrystalline cellulose and especially to an environmentally friendly and resource-saving method for producing fractionally homogeneous compositions based on microcrystalline cellulose.

### BACKGROUND OF THE INVENTION

Microcrystalline cellulose (MCC) containing compositions is a commodity useful for pharmaceuticals (tabletting aid), foods (fat substitute), cosmetics (gel-former), chemistry (catalyst's carrier), biotechnology (substrate), polymer composites (filler) and other fields. MCC is currently produced by hydrolysis of cellulose materials by mineral acids or acidic salts at elevated temperatures and sometimes at heightened pressures. Typical of MCC commercially available compositions are composed of functional ingredients, such as surfactants, wetting agents, dispersants, binders, gel-formers, gums, fillers, pigments, extenders, salts and others.

Known methods for producing MCC-containing compositions include the steps of hydrolysis of the cellulose raw materials; dilution and separation of the destroyed product by filtering or centrifugation; neutralization; washing; collection of MCC wet-cake by filtering or centrifugation; mixing of the wet-cake with various ingredients; homogenization of the composition; and then, optionally, drying and pulverization.

A process for preparation compositions of MCC and calcium carbonate is disclosed in U.S. Pat. No. 4,744,987, providing a process wherein a hydrolyzed and washed wet-cake of MCC is separated and its up to 20% aqueous dispersion is well mixed with slurry of calcium carbonate, and then dry-pulverized to obtain of a particulate product having particle size from about 20 to 150 microns.

U.S. Pat. No. 6,037,380 describes process of obtaining compositions of MCC comprises separation of the MCC wet-cake after cellulose hydrolysis and washing, mixing of the wet-cake with various functional ingredients—insoluble attiring aids, protective colloids, etc., blending and wet grinding at a high shear for preparation settling stable water dispersions.

U.S. Pat. No. 6,541,627 discloses a method of producing dispersed cellulose composition comprising the following steps: dissolving of cellulose; precipitation of cellulose flocs from solutions; hydrolysis of the flocs; and washing of cellulose micro-particles to remove of the acid; then cellulose particles are dispersed together with various substances, such as binders, surfactants, inorganic salts, pigments, etc.

The hydrolysis step for manufacturing of MCC and its based compositions is performed in acidic medium using solutions of strong mineral acids (usually, hydrochloric and sulfuric) at increased temperatures (e.g., 100 to 130° C.) and different acid/cellulose weight ratios (e.g. 10 to 20). U.S. Pat. No. 6,495,190 describes a process wherein the hydrolysis of cellulose is carried out with 7% HCl at 105° C. The destroyed acidic cellulose material is further diluted with water, collected by filtration, washed on the filter up to neutral pH and filtered to obtain an acid-free wet-cake. In a subsequent step, wet-cake of MCC is mixed and homogenized with water-soluble polysaccharides or gums by means of wet grinder, attrition mill and other grinding equipment and dry-pulverized to prepare products having average particle size of about 30 microns.

Aforesaid available methods are characterized by significant disadvantages, such as that neutralization and washing of the acidic hydrolyzed cellulose and preparation fully acid-free wet-cake results in an unavoidable damage to the environment, especially due to the formation high volume of the polluted wastewater. As it is follows, for example, from U.S. Pat. No 4,391,973, in the case that cellulose hydrolysis is provided by a means of admixing 9% HCl and at acid/cellulose ratio 20, the volume of the produced wastewater is about 100 liter per one kg cellulose; wherein the wastewater is polluted with about 20 g/l inorganic and organic contaminants. Regeneration of the wastewater demands high consumption of chemicals and energy that rise production costs beyond cost effectiveness. Moreover, existing methods essentially involve the full loss of reagents and also partial loss of cellulose raw materials and thus leads to decreased MCC productivity,—usually about 80 to 85% yield.

Another disadvantage of the existing methods for production homogeneous MCC is their requirements for complicated mills or multiple grinding equipment, which oblige long, high-energy and expensive mechanical treatment. Thus a need for cost effective methods involving simple mechanical equipment and processing is highly needed for this purpose.

### SUMMARY OF THE INVENTION

It is the object of the invention to present an environmental friendly method for the production of fractionally homogeneous compositions of microcrystalline cellulose (MCC). This cost effective method consists of the following steps: hydrolyzing cellulose-containing raw material with a catalytic system comprising at least one acidic catalyst in the presence of at least one process additive at a 0.1 to 10 catalytic system/cellulose weight ratio; neutralizing said acid with one or more precipitator in the manner that fine particles of insoluble ingredients precipitate into a MCC containing slurry; admixing at least one modifier; and then homogenizing of the composition so that a MCC product characterized by uniformly dispersed micro-particle cellulose and various functional ingredients is obtained.

It is in the scope of the present invention wherein the aforesaid acidic catalyst is a mineral acid and/or wherein the process additive is selected from acidic stable non-ionic wetting agent, such as polyalkylenoxide polysiloxane or any of its derivatives, oxidant or any mixture thereof. According to one embodiment of the present invention, the concentration of the non-ionic wetting agent ranges from 0.02 to 0.20% (weight percent); or specifically 0.05 to 0.10% according to yet another embodiment.

It is also in the scope of the present invention wherein the aforesaid oxidant is selected from potassium permanganate, hydrogen peroxide, sodium and/or potassium peroxides or any mixture of the peroxides. According to one embodiment of the present invention, the concentration of oxidant ranges from 0.5 to 5.0% (weight percent); or specifically from 1.0 to 5.0% (weight percent).

It is also in the scope of the present invention wherein the aforesaid catalytic system comprising the acidic catalyst and process additive to cellulose weight ratio ranges from 0.5 to 5.0; or specifically from 2.5 to 3.5.

It is also in the scope of the present invention wherein the aforesaid precipitator is selected from low-soluble carbonates, hydroxides and oxides, soluble basic silicates, salts of carbonic and fatty acids or any mixture thereof. Said low-soluble precipitators comprise cations of calcium, barium or a combination thereof. The salts of carbonic acids may contain radicals of low- or insoluble organic acids, for example capric acid, or any mixture thereof. The salts of fatty acids are based on stearic acid, palmitic acid, oleic acid or any mixture thereof. The precipitator may also comprise one or more of the specific constituents that are characteristic of fillers, pigments, anti-blocking agents; lubricants; rheology adjusters or any mixture thereof. The said precipitator is preferably admixed to the acidic MCC-slurry so that a neutral pH in the range of 6 to 8 is obtained.

It is also in the scope of the present invention wherein the aforesaid modifier is selected from thickeners, dispersers, anti-foaming agents, emulsifiers, preservatives, biocides, pigments or any mixture thereof. The aforementioned thickeners and/or dispersers are preferably admixed so 5 to 20%, or preferably 8 to 10% (weight percent) mixture is obtained. The said preservatives and/or biocides are admixed so 0.1 to 2.0%, or preferably 0.5 to 1.0% (weight percent) mixture is obtained.

It is still in the scope of the present invention wherein the MCC product comprising solid content of 1 to 50% or specifically 10 to 30% (weight percent). Lastly, said method may additionally comprise of a final step of spray drying of the uniformly dispersed micro-particle cellulose compositions.

It is also in the scope of the present invention to provide an MCC product characterized by uniformly dispersed micro-particle cellulose produced by a method as defined in any of the above.

### DETAILED DESCRIPTION OF THE INVENTION

The following description is provided, alongside all chapters of the present invention, so as to enable any person skilled in the art to make use of said invention and sets forth the best modes contemplated by the inventor of carrying out this invention. Various modifications, however, will remain apparent to those skilled in the art, since the generic principles of the present invention have been defined specifically to provide a novel, environment-friendly and resource-saving method for the production of fractionally homogeneous compositions of microcrystalline cellulose (MCC). This method comprises of the following steps: (a) hydrolyzing cellulose-containing raw material with at least one acidic catalyst in the presence of at least one process additive at a low catalytic system/cellulose ratio; (b) neutralizing said acid with one or more precipitator in the manner that fine particles of insoluble ingredients precipitate into a MCC containing slurry; (c) admixing at least one modifier; and then (d), homogenizing of the composition so that a MCC product characterized by a uniformly dispersed micro-particle cellulose and various functional ingredients is obtained.

The term “cellulose-containing raw material” refers in the present teaching of the invention to any material, mixture or composition that contains cellulose. More specifically, said term refers to a material selected in a non-limiting mariner from chemical-grade and paper-grade wood celluloses, including sulfite, sulfate and prehydrolized kraft, bleached and non-bleached celluloses from hardwood and softwood; chemical and special paper-grade cotton celluloses; natural and artificial cellulose fibers; as well as recycled paper and cardboard; scrap of paper, cardboard and cellulose-based textiles materials, etc.

The hydrolysis step that is described in the present invention is provided inter alia by contacting or admixing cellulose-containing raw material with sufficient amount of acid; particularly mineral acid or a mixture of two or more mineral acids. More specifically, said mineral acid may be selected in a non-limiting manner from one or more acids, particularly from hydrochloric, sulfuric, nitric and phosphoric acid. The aforesaid hydrolysis step may be performed according to one embodiment of the present invention using acid with a concentration of 0.1 to 2.5N. According to yet another embodiment of the present invention, the hydrolysis is performed at a temperature range of 80 to 150° C. for a time period lasting from about 15 to 60 min.

It is acknowledged in this respect that such a hydrolysis may result by a chemical splitting of the chains in amorphous regions of cellulose microfibrils and release of linked microcrystallites. Some process additives, such as acid-stable non-ionic wetting agents and oxidants are thus presented to the acid catalysts at the hydrolysis step. The process additives may be selected from polyalkylenoxide polysiloxanes or any of its derivatives (e.g. commercially available Dow Corning No.193 or Abil B8851 products), while hydrogen peroxide, sodium or potassium peroxide and potassium permanganate can be selected as oxidants.

The term ‘wetting agents’ refers according to the present invention to any chemical suitable or adapted to provide a uniform and prompt penetration of acid catalyst and/or oxidant into cellulose, so residual lignin and hemicelluloses are destructed and accessibility of cellulose microfibrils to reagents is ensured. The concentration of the wetting agent in the acid preferably ranges from 0.02 to 0.2% and of the oxidant similarly ranges from 1 to 5%. Mixture of said mineral acid and said process additives is hereinafter termed ‘catalytic system’. According to another embodiment of the present invention, the hydrolysis step is provided at a low catalytic system/cellulose ratio of 0.5 to 5, and at preferable ratio of 2.5 to 3.5.

After obtaining a significant chemical splitting of the cellulose-containing raw material provided by the aforementioned acid hydrolysis, one or more precipitators are introduced for the neutralization of said acid, in the manner that fine particles of water-insoluble ingredients precipitating into MCC-slurry are obtained. These precipitators are selected in a non-limited manner from carbonates, oxides and hydroxides of calcium and barium; soluble basic silicates, e.g. water glass; salts of fatty acids, e.g., sodium stearate or palmitate, etc. These insoluble ingredients have various functions, which are characteristic of a filler (e.g., calcium sulfate), pigment (e.g., barium sulfate), anti-blocking agent and lubricant (e.g., stearin, palmitic acid), rheology adjuster (e.g., silica gel), etc.

Various modifiers are then admixed to the slurry of MCC and insoluble ingredients. These modifiers are selected from thickeners, dispersers and preservatives. The term ‘thickener’ is referring according to the present invention to any agent adapted for increasing the viscosity of the liquid composition thus ensuring optimal dispersing condition and settling stability of the end product. This thickener can be selected from soluble cellulose derivatives, e.g. methylcellulose, hydroxyethyl cellulose, etc; xanthan gum, alginates, polyvinyl alcohol and some others. The term ‘disperser’ is referring according to the present invention to any agent adapted to facilitate disaggregation and distribution of the solid particles and to improve homogeneity of the compositions. The disperser may be selected from tri-sodium polyphosphate (TSPP), low molecular sodium polyacrylate (SPA), hydrophilic derivatives of fatty acids such as the commercially available product Tego Dispers 740 W (TD) and others.

The term ‘preservative’ refers according to the present invention to any agent or biocide adapted to prevent biodegradation of the wet cellulose-based dispersions and ensure that end product has a long shelf life. Such a preservative may be selected in a non-limiting manner from methyl paraben, potassium sorbate or chlorhexidine gluconate. The amount of the thickeners and dispersing agents in the composition can be from about 5 to 20%, while of the preservative from 0.1 to 2%. In is aclnowledged in this respect that the teem about is generally referring to the range of 20%.

The compositions of MCC and various functional ingredients having concentrations 1 to 30%, preferable 2 to 10%, are homogenized using any existing equipment, like blender, homogenizer, attrition mill, etc.

In order to concentrate of the compositions up to 40-60% solid content, any existing technology, for instance high-pressure filtration, high-speed centrifugation or vacuum evaporation, can be used. To obtain a dry powder, the wet MCC-containing composition can be dried according to methods well known in the art such as spray, fluid bed drying, etc.

### EXAMPLES

Various examples were carried out to prove the embodiments claimed in the present invention. Some of these experiments are referred hereinafter. The examples describe the manner and process of the present invention and set forth the best mode contemplated by the inventors for carrying out the invention, but are not to be construed as limiting the invention.

The chemical grade bleached prehydrolyzed craft and paper grade non-bleached kraft celluloses available from International Paper Co. were used for illustration of the invention. Crystallinity degree was determined using Rigaku-Ultima Plus X-ray diffractometer. Particle size distribution and exterior average specific surface (S) were studied by a means of a Mastersizer 2000 Apparatus of Malvern Instrument Ltd. Average particle size (d_{0.5}), width of the particle distribution curve (W=d_{0.75}−d_{0.25}) and heterogeneity parameter (H); wherein H value equal W/d_{0.5} of the homogenized composition; were determined. Yield of the solid products, volume of the wastewater and loss of the acidic catalyst also were measured.

### Example 1

80 g of bleached prehydrolyzed kraft cellulose was cut on pieces of 1 to 3 cm that were putted in a 500-ml laboratory glass reactor positioned in a silicon oil bath. Catalytic system containing 10% of sulfuric acid, 1% of hydrogen peroxide and 0.05% of Dow Corning 193 wetting agent was added to the reactor so as obtaining catalytic system/cellulose ratio 3.5. The reactor was closed hermetically with a cover, heated up to 105° C., kept at this temperature for 30 min and then cooled. The paste-like slurry of MCC was transferred to glass beaker fitted with a glass stirrer rod and about 100 g of 29% water dispersion of calcium carbonate is slowly added to acidic MCC-slurry at a stirring in order to neutralize the acid and precipitate particles of the insoluble ingredient—calcium sulfate. Then 50 g of 2% hydroxyethyl cellulose as a thickener, 20 g of 1% SPA and 10 g of 5% TSPP as dispersers and 4 g of 0.02% methyl paraben as a preservative were introduced to the composition at a stirring. The composition had 24% solid content. In order to increase solid concentration to 50%, the composition was evaporated by vacuum drier at 75 to 80° C.

About 100 g of the composition was washed with deionized water and absolute ethanol using the porcelain Buchner funnel containing a Whatman filter paper No 42, filtered at a vacuum, dried in the air-dryer at 100° C. to constant weight and weighed in order to measure the solid yield. Other portion of the composition was diluted with water to 5% solid concentration and homogenized by a Waring blender at 20,000 rpm for 15 min.

### Example 2

The same procedure as it was described in the example 1, wherein 100 g of 21.3% water dispersion of calcium hydroxide was added to acidic MCC-slurry in order to neutralize the acid and precipitate particles of the insoluble ingredient.

### Example 3

80 g of bleached prehydrolyzed kraft cellulose was cut on pieces of 1 to 3 cm that were putted in a 500-ml laboratory glass reactor positioned in a silicon oil bath. Catalytic system containing 7% of sulfuric acid, 1% of potassium permanganate and 0.05% of Dow Corning 193 wetting agent was added to the reactor so as obtaining catalytic system/cellulose ratio 3.5. The reactor was closed hermetically with a cover, heated up to 110° C., kept at this temperature for 30 min and then cooled. The paste-like slurry of MCC was transferred to glass beaker fitted with a glass stirrer rod and about 200 g of 17.5% water dispersion of barium hydroxide is slowly added to acidic MCC-slurry at a stirring in order to neutralize the acid and precipitate barium sulfate. Then 60 g of 2% polyvinyl alcohol as a thickener, 20 g of 1% SPA and 10 g of 5% TSPP as dispersers and 5 g of 0.01% clilorhexidine gluconate as a preservative were introduced to the composition at a stirring.

About 100 g of the composition was filtered at a vacuum through porcelain Buchner funnel containing a Whatman filter paper No. 42, washed with water and absolute ethanol, filtered once again, dried in the air-dryer at 100° C. to constant weight and weighed in order to measure the solid yield. Other portion of the composition was diluted with water to 10% solid concentration and homogenized by Waring blender at 20,000 rpm for 15 min.

To obtain a dry powder having average particle size about 20 microns, the 10% dispersion was spray dried at the inlet temperature 108° C. and outlet temperature 110° C.

### Example 4

80 g of bleached prehydrolyzed kraft cellulose was cut on pieces of 1 to 3 cm and mixed with 60 g of 50% H₂SO₄ in a ceramic blender by means of glass balls. The impregnated cellulose was putted in a 500 ml laboratory glass reactor positioned in a silicon oil bath. The reactor was closed hermetically with a cover and heated at 80° C. for 20 min. Then 260 g of 0.05% Dow Corning 193 was added to the catalyst so as obtaining catalytic system/cellulose ratio about 4. The reactor is heated up to 110° C., kept at this temperature for 30 min and then cooled. The paste-like slurry of MCC was transferred to glass beaker fitted with a glass stirrer rod and about 200 g of 2% sodium stearate and then about 60 g of 50% water dispersion of calcium carbonate is slowly added to acidic MCC-slurry at a stirring in order to neutralize the acid and precipitate stearin and calcium sulfate. Then 50 g of 2% methyl cellulose as a thickener, 15 g of 1% SPA and 10 g of 5% TSPP as dispersers and 4 g of 0.03% potassium sorbate as a preservative were introduced to the composition at a stirring.

About 10 g of the composition was filtered at a vacuum through porcelain Buchner funnel containing a Whatman filter paper No. 42, washed with deionized water and absolute ethanol, filtered once again, dried in the air-dryer at 100° C. to constant weight and weighed in order to measure the solid yield. Other part of the composition was diluted with water to 2% solid concentration and homogenized by a Waring blender at 20,000 rpm for 15 min.

### Example 5

The same procedure as it was described in the example 1, wherein paper grade non-bleached cellulose was used.

### Example 6

80 g of bleached prehydrolyzed kraft cellulose was cut on pieces of 1 to 3 cm that were putted in a 500-ml laboratory glass reactor positioned in a silicon oil bath. Catalytic system containing 7% of sulfuric acid, 5% of phosphoric acid and 0.05% Dow Corning 193 wetting agent was added to the reactor so as obtaining catalytic system/cellulose ratio 3.5. The reactor was closed hermetically with a cover, heated up to 110° C., kept at this temperature for 30 min and then cooled. The paste-like slurry of MCC was transferred to glass beaker fitted with a glass stirrer rod and about 100 g of 2% sodium palmitate and 100 g of 38% water dispersion of calcium hydroxide is slowly added to acidic MCC-slurry at a stirring in order to neutralize the acid and precipitate particles of insoluble ingredients—palmitic acid, calcium sulfate and calcium phosphate. Then 50 g of 2% polyvinyl alcohol as a thickener, 20 g of 1% TD and 10 g of 5% TSPP as dispersers and 5 g of 0.02% methyl paraben as a preservative were introduced to the composition at a stirring.

About 100 g of the composition was filtered at a vacuum through porcelain Buchner funnel containing a Whatman filter paper No. 42, washed with deionized water and absolute ethanol, filtered once again, dried in the air-dryer at 100° C. to constant weight and weighed in order to measure the solid yield. Other portion of the composition was diluted with water to 2.5% solid concentration and homogenized by a Waring blender at 20,000 rpm for 15min.

### Example 7

80 g of bleached prehydrolyzed kraft cellulose was cut on pieces of 1-3 cm that were putted in a 500-ml laboratory glass reactor positioned in a silicon oil bath. Catalytic system containing 7% of hydrochloric acid, 1% of sodium peroxide and 0.05% of Abil B8851 wetting agent was added to the reactor so as obtaining catalytic system/cellulose ratio 3. The reactor was closed hermetically with a cover, heated up to 110° C., kept at this temperature for 30 min and then cooled. The paste-like slurry of MCC was transferred to glass beaker fitted with a glass stirrer rod and about 20% water glass is slowly added to acidic MCC-slurry at a stirring in order to neutralize the acid and precipitate silica gel. Then 50 g of 2% hydroxyethyl cellulose as a thickener and 25 g of 1% SPA as a disperser and 4 g of 0.02% methyl paraben as a preservative were introduced to the composition at a stirring.

About 100 g of the composition was filtered at a vacuum through porcelain Buchner funnel containing a Whatman filter paper No. 42, washed with deionized water and absolute ethanol, filtered once again, dried in the air-dryer at 100° C. to constant weight and weighed in order to measure the solid yield. Other portion of the composition was diluted with water to 1% solid concentration and homogenized by Waring blender at 20,000 rpm for 15min.

### Example 8

The same procedure as it was described in the example 7, wherein homogenization of the composition was carried out by Gaulin homogenizer at 100 MPa for 15 to 20 min.

### Example 9

The same procedure as it was described in the example 7, wherein 5% of nitric acid was used at the hydrolysis step.

### Example 10

80 g of bleached prehydrolyzed kraft cellulose was cut on pieces of 1-3 cm that were putted in a 500-ml laboratory glass reactor positioned in a silicon oil bath. Catalytic system containing 5% of hydrochloric acid and 0.05% of Dow Corning 193 wetting agent was added to the reactor so as obtaining catalytic systen/cellulose ratio 3. The reactor was closed hermetically with a cover, heated up to 115° C., kept at this temperature for 30 min and then cooled. The paste-like slurry of MCC was mixed with 200 ml water and filtered at a vacuum through porcelain Buchner funnel containing Whatman filter paper No. 42. The filter wet-cake was transferred to glass beaker and about 20% water glass was slowly added at a mild stirring in order to neutralize the acid and precipitate silica gel. Then 50 g of 2% methyl cellulose as a thickener and 25 g of 1% SPA as a disperser and 4 g of 0.03% methyl paraben as a preservative were introduced to the composition at a stirring.

About 100 g of the composition was filtered at a vacuum through porcelain Buchner funnel containing Whatman filter paper No. 42, washed with deionized water and absolute ethanol, filtered once again, dried in the air-dryer at 100° C. to constant weight and weighed in order to measure the solid yield. Other portion of the composition was diluted with water to 1% solid concentration and homogenized by Waring blender at 20,000 rpm for 15 min.

### Example 11

The same procedure as it was described in the example 7, wherein paper grade non-bleached cellulose was used.

### Comparative Example 12

100 g of bleached prehydrolyzed kraft cellulose was cut on pieces of 1 to 3 cm that were mixed with 170 g water and disintegrated by Waring blender at 20,000 rpm for 5 min to obtain 37% cellulose pulp. This pulp is putted in a 3000 ml glass beaker fitted with a glass stirrer rod and positioned in a silicon oil bath. About 1,800 g of 10% hydrochloric acid was added to the pulp at a mild stirring. The reaction system was stirred, heated up to 105° C. and kept at this temperature for 15 min. The acidic MCC slurry was poured into 2000 ml water at room temperature at a stirring and then filtered at a vacuum through porcelain Buchner funnel containing Whatman filter paper No. 42. The filter cake was washed with about 800 g water to pH 6, neutralized with 1% ammonium hydroxide and washed additionally with about 800 g deionized water to remove the ammonium chloride and the MCC was filtered once again to prepare wet-cake having pH 7 and MCC content of about 40%.

### Comparative Example 13

The same procedure as it was described in the comparative example 12, wherein paper grade non-bleached cellulose was used.

### Comparative Example 14

About 100 g of 40% MCC wet-cake were prepared in accordance with the comparative example 12. This amount of the MCC wet-cake was putted into 1000 ml glass, mixed with 400 g of 5.5% water dispersion of calcium carbonate at a mild stirring in order to obtain the composition. About 100 g of the composition was filtered, washed with deionized water and absolute ethanol, dried in the air-dryer at 100° C. to constant weight and weighed in order to measure the solid yield. Other portion of the composition was diluted with water to 5% solid concentration and homogenized by Waring blender at 20,000 rpm for 15 min.

### Comparative Example 15

About 100 g of 40% MCC wet-cake were prepared in accordance with the comparative example 13. This amount of the MCC wet-cake was putted into 1000 ml glass, mixed with 400 g of 5.5% water dispersion of calcium carbonate at a mild stirring in order to obtain the composition. About 100 g of the composition was filtered, washed with deionized water and absolute ethanol, dried in the air-dryer at 100° C. to constant weight and weighed in order to measure the solid yield. Other portion of the composition was diluted with water to 5% solid concentration and homogenized by Waring blender at 20,000 rpm for 15 min.

### Comparative Example 16

The same procedure as it was described in the comparative example 14, wherein calcium sulfate was added to MCC wet-cake.

### Comparative Example 17

The same procedure as it was described in the comparative example 14, wherein calcium phosphate was added to MCC wet-cake.

### Comparative Example 18

The same procedure as it was described in the comparative example 14, but silica was added to MCC wet-cake.

### Comparative Example 19

The same procedure as it was described in the comparative example 18, wherein homogenization of the composition was carried out by Gaulin homogenizer at 100 MPa for 15 to 20 min.

The testing results of the examples performed in accordance with the present invention are shown in Table 1, while testing results of the comparative examples performed in accordance with the existing methods are shown in Table 2.

TABLE 1
Testing results of the compositions prepared according to present invention

| | | | Volume of | Loss of acidic | | | |
|---|---|---|---|---|---|---|---|
| Example | | Yield of | wastewater, | catalyst, | d_{0.5}, | W, | |
| No | Solid composition | solid, % | l/kg solid | kg/kg solid | microns | microns | S × 10⁻³ m²/kg |
| 1 | MCC - 67%, | 97.3 | 0 | 0 | 9.1 | 10.5 | 1.3 |
| | CaSO₄ - 33% | | | | | | |
| 2 | MCC - 67%, | 97.2 | 0 | 0 | 9.7 | 11.6 | 1.2 |
| | CaSO₄ - 33% | | | | | | |
| 3 | MCC - 67%, | 97.8 | 0 | 0 | 10.2 | 12.3 | 1.1 |
| | BaSO₄ - 33% | | | | | | |
| 4 | MCC - 65%, | 96.4 | 0 | 0 | 5.3 | 5.6 | 1.8 |
| | CaSO₄ - 32%, | | | | | | |
| | Stearin - 3% | | | | | | |
| 5 | MCC - 67%, | 95.2 | 0 | 0 | 11.3 | 13.7 | 1.0 |
| | CaSO₄ - 33% | | | | | | |
| 6 | MCC - 61%, | 97.6 | 0 | 0 | 11.8 | 14.0 | 1.0 |
| | CaSO₄ - 21%, | | | | | | |
| | Ca₃(PO₄)₂- 17% | | | | | | |
| | Palmitic acid - 1% | | | | | | |
| 7 | MCC - 70%, | 96.2 | 0 | 0 | 5.0 | 5.5 | 1.9 |
| | SiO₂ - 30% | | | | | | |
| 8 | MCC - 70%, | 96.0 | 0 | 0 | 0.6 | 0.75 | 16.3 |
| | SiO₂ - 30% | | | | | | |
| 9 | MCC - 65%, | 97.6 | 0 | 0 | 6.5 | 6.8 | 1.5 |
| | SiO₂ - 35% | | | | | | |
| 10 | MCC - 87%, | 94.1 | 3.4 | 0.07 | 10.2 | 11.0 | 1.1 |
| | SiO₂ - 13% | | | | | | |
| 11 | MCC - 70%, | 95.0 | 0 | 0 | 6.8 | 7.4 | 1.4 |
| | SiO₂ - 30% | | | | | | |

TABLE 2
Testing results of the compositions prepared according to existing methods

| | | | Volume of | Loss of acidic | | | |
|---|---|---|---|---|---|---|---|
| Comparative | Solid | | wastewater | catalyst, | d_{0.5}, | W, | |
| Example No. | composition | Yield of solid, % | l/kg solid | kg/kg solid | microns | microns | S × 10⁻³ m²/kg |
| 14 | MCC - 65%, | 88.2 | 24.3 | 1.35 | 13.2 | 19.8 | 0.73 |
| | CaCO₃ - 35% | | | | | | |
| 15 | MCC - 65%, | 86.0 | 24.8 | 1.38 | 15.4 | 23.1 | 0.63 |
| | CaCO₃ - 35% | | | | | | |
| 16 | MCC - 65%, | 88.1 | 24.3 | 1.35 | 13.8 | 20.4 | 0.70 |
| | CaSO₄ - 35% | | | | | | |
| 17 | MCC - 65%, | 88.3 | 24.3 | 1.35 | 13.4 | 19.6 | 0.72 |
| | Ca3(PO₄)₂ - 35% | | | | | | |
| 18 | MCC - 65%, | 88.0 | 24.3 | 1.35 | 14.3 | 20.7 | 0.68 |
| | SiO₂ - 35% | | | | | | |
| 19 | MCC - 65%, | 88.1 | 24.3 | 1.35 | 1.2 | 2.5 | 7.6 |
| | SiO₂ - 35% | | | | | | |

Heterogeneity parameter H of some compositions obtained in accordance with the present and existing methods is shown in Table 3.

TABLE 3
Heterogeneity parameter H of some MCC-based compositions

| | | W, | | |
|---|---|---|---|---|
| Example No | Solid composition | microns | d_{0.5}, microns | H |
| 1 | MCC - 67%, | 10.5 | 9.1 | 1.1 |
| (The present method) | CaSO₄ - 33% | | | |
| 14 | MCC - 65%, | 19.8 | 13.2 | 1.5 |
| (The existing method) | CaSO₄ - 35% | | | |
| 7 | MCC - 70%, | 5.5 | 5.0 | 1.1 |
| (The present method) | SiO₂ - 30% | | | |
| 18 | MCC - 65%, | 20.7 | 14.3 | 1.5 |
| (The existing method) | SiO₂ - 35% | | | |
| 8 | MCC - 70%, | 0.75 | 0.6 | 1.25 |
| (The present method | SiO₂ - 30% | | | |
| 19 | MCC - 65%, | 2.5 | 1.2 | 2.1 |
| (The existing method) | SiO₂ - 35% | | | |

Table 1 and 2 evidently shows that the present invention provides for a resource-saving method, which provides increasing yield on about 8-9%, in comparison with existing methods and reducing or fill eliminating loss of an acidic catalyst. The present invention permits producing fractionally homogeneous MCC-based composition having lower heterogeneity (H-value is 1 to 1.3, instead 1.4 to 2.1 for existing methods), more developed particle surface (more than 1000 m²/kg, instead less 1000 m²/kg for the existing methods), finer particles and narrow distributions of particle size (width W of the distribution curve is 40 to 70% narrower) than the existing methods at the same homogenization equipment and conditions (Table 1-3). Moreover, the present invention is environmentally-friendly, because it permits the production of MCC-containing compositions with minimal amount or without forming of the wastewater.

As it follows from the testing results, the MCC-based compositions obtaining in accordance with present invention are characterizing with micron- or submicron-scale particles, uniform fractional composition having heterogeneity H-parameter 1 to 1.3, developed external specific surface more 1000 m²/kg and high crystallinity of the solid phase, 85 to 90%.

1. A method for the production of fractionally homogeneous compositions containing microcrystalline cellulose (MCC), comprising the steps:
(a) hydrolyzing cellulose-containing raw material with a catalytic system comprising at least one acidic catalyst in the presence of at least one process additive at about 0.1 to 10 catalytic system/cellulose weight ratio;
(b) neutralizing said acid with one or more precipitator in the manner that fine particles of insoluble ingredients precipitate into a slurry containing MCC;
(c) admixing at least one modifier; and
(d) homogenizing of the composition so that a MCC product characterized by a uniformly dispersed micro-particle cellulose material and various functional ingredients is obtained.

2. The method according to claim 1, wherein the acidic catalyst is a mineral acid.

3. The method according to claim 1, wherein the process additive is selected from acidic stable non-ionic wetting agent, oxidant or any mixture thereof.

4. The method according to claim 3, wherein the non-ionic wetting agent is a polyalkylenoxide polysiloxane or any of its derivatives thereof

5. The method according to claim 3, wherein the concentration of the non-ionic wetting agent is in the range of about 0.02 to 0.20% (weight percent).

6. The method according to claim 3, wherein the concentration of the non-ionic wetting agent is in the range of about 0.05 to 0.10% (weight percent).

7. The method according to claim 3, wherein the oxidant is selected from potassium permanganate, hydrogen peroxide, sodium and/or potassium peroxides or any peroxide-containing mixture.

8. The method according to claim 3, wherein the concentration of oxidant is in the range of bout 0.5 to 5.0% (weight percent).

9. The method according to claim 3, wherein the concentration of oxidant is in the range of about 1.0 to 5.0% (weight percent).

10. The method according to claim 1, wherein the catalytic system comprising the acidic catalyst and process additive to cellulose weight ratio is in the range of about 0.5 to 5.0.

11. The method according to claim 1, wherein the catalytic system comprising the acidic catalyst and process additive to cellulose weight ratio is in the range of about 2.5 to 3.5.

12. The method according to claim 1, wherein the precipitator is selected from low-soluble carbonates, hydroxides and oxides, soluble basic silicates, salts of carbonic and fatty acids or any mixture thereof.

13. The method according to claim 12, wherein the salts of fatty acids are based on stearic acid, palmitic acid, oleic acid or any mixture thereof.

14. The method according to claim 12, wherein the precipitator also comprising one or more of the specific constituents that are characteristic of fillers, pigments, anti-blocking agents; lubricants; rheology adjusters or any mixture thereof.

15. The method according to claim 12, wherein the low-soluble precipitators comprise cations of calcium, barium or a combination thereof.

16. The method according to claim 1, wherein the precipitator is admixed to the acidic MCC-slurry so that a neutral pH of about 6 to 8 is obtained.

17. The method according to claim 1, wherein the modifier is selected from thickeners, dispersers, emulsifiers, anti-foaming agents, preservatives, biocides, pigments or any mixture thereof.

18. The method according to claim 1, wherein thickeners and/or dispersers are admixed such that a about 5 to 20% (weight percent) mixture is obtained.

19. The method according to claim 1, wherein thickeners and/or dispersers are admixed such that about 8 to 10% (weight percent) mixture is obtained.

20. The method according to claim 1, wherein preservatives and/or biocides are admixed such that about 0.1 to 2.0% (weight percent) mixture is obtained.

21. The method according to claim 1, wherein preservatives and/or biocides are admixed such that about 0.5 to 1.0% (weight percent) mixture is obtained.

22. The method according to claim 1, wherein the MCC product comprising solid content of about 1 to 50%.

23. The method according to claim 1, wherein the MCC product comprising solid content of about 10 to 30%.

24. The method according to claim 1, additionally comprising the step of spray-drying the uniformly dispersed micro-particle cellulose materials.

25. An MCC product characterized by uniformly dispersed micro-particle cellulose produced by a method comprising the following steps:
(a) hydrolyzing cellulose-containing raw material with a catalytic system comprising at least one acidic catalyst in the presence of at least one process additive at about 0.1 to 10 catalytic system/cellulose weight ratio;
(b) neutralizing said acid with one or more precipitator in the manner that fine particles of insoluble ingredients participates into a MCC containing slurry;
(c) admixing at least one modifier, and
(d) homogenizing of the composition so, that an obtained MCC product are characterizing with micron- or submicron-scale particles; uniform fractional composition having heterogeneity H-parameter of about 1 to 1.3, developed external specific surface more 1000 m²/kg and high crystallinity of the solid phase, of about 85 to 90%.
